(19) United States
(12) Patent Application Publication
(10) Pub. No.: US 2020/0060557 A1
(43) Pub. Date: Feb. 27, 2020

(54) CONTROL APPARATUS, CONTROL SYSTEM, CONTROL METHOD, AND PROGRAM

(71) Applicant: SONY CORPORATION, TOKYO (JP)

(72) Inventors: TAKESHI MATSUI, TOKYO (JP); TETSURO KUWAYAMA, TOKYO (JP); HIROSHI YOSHIDA, KANAGAWA (JP); ISAMU NAKAO, KANAGAWA (JP); FUMISADA MAEDA, TOKYO (JP); GORO FUJITA, KANAGAWA (JP); AKIO FURUKAWA, TOKYO (JP)

(21) Appl. No.: 16/609,799

(22) PCT Filed: Mar. 20, 2018
(86) PCT No.: PCT/JP2018/010984

§ 371 Date: Oct. 31, 2019

(30) Foreign Application Priority Data
May 9, 2017 (JP) 2017-093013

(51) Int. Cl. A61B 5/026; G02B 21/00; A61B 5/00

(52) U.S. Cl. CPC A61B 5/0261; G02B 21/0028; A61B 5/0042; G02B 21/008; A61B 5/0066; G02B 21/0032

(57) ABSTRACT

A control apparatus according to an aspect of the present technology includes a signal generation section. The signal generation section generates speckle data on the basis of an image signal of a subject imaged by using laser light as illumination, and generates a control signal for controlling output from a laser light source that emits the laser light on the basis of the generated speckle data.

### TECHNICAL FIELD

The present technology relates to a control apparatus, a control system, a control method, and a program that are applicable to observation and the like of a living tissue.

### BACKGROUND ART

Patent Literature 1 discloses an analysis device that analyzes a blood flow or the like of a living tissue on the basis of speckle data obtained through emission of laser light. In this analysis device, an image forming optical system forms an image of the laser light emitted to an analysis target, and an imaging device captures a speckle image. Numerical aperture of the image forming optical system is controlled on the basis of speckle contrast calculated on the basis of the speckle image. This makes it possible to increase the speckle contrast, and this makes it possible to enhance measurement accuracy of the blood flow or the like (see paragraph [0056], FIG. 1, FIG. 6, and the like of Patent Literature 1)

### CITATION LIST

### Patent Literature

Patent Literature 1: JP 2016-5525A

### DISCLOSURE OF INVENTION

### Technical Problem

As described above, technologies capable of exhibiting high accuracy are desired for observation or the like of a living tissue using speckle data.

In view of the circumstances as described above, a purpose of the present technology is to provide the control apparatus, the control system, the control method, and the program that are capable of observing a living tissue or the like with high accuracy.

### Solution to Problem

In order to achieve the above-mentioned purpose, a control apparatus according to an aspect of the present technology includes a signal generation section. The signal generation section generates speckle data on the basis of an image signal of a subject imaged by using laser light as illumination, and generates a control signal for controlling output from a laser light source that emits the laser light on the basis of the generated speckle data.

The control apparatus is configured to control output from the laser light source on the basis of speckle data computed from an image signal of a subject. This makes it possible to suppress variation in the output from the laser light source and observe a living tissue or the like with high accuracy.

The subject may include an observation target and a standard sample for calibration, and the signal generation section may generate the speckle data on the basis of an image signal of the standard sample.

This makes it possible to detect the output from the laser light source or its variation with high accuracy.

The control apparatus may further includes a display control section that causes the display section to display a speckle contrast image of the subject.

For example, this makes it possible to observe presence or absence of blood vessels or the like with high accuracy.

In the case where the speckle data is less than a first threshold, the signal generation section may be configured to generate a control signal for increasing or decreasing the output from the laser light source in a manner that the speckle data becomes the first threshold or more.

This makes it possible to control the laser light source in a manner that a desired observation image is obtained from its output.

The signal generation section may be configured such that: in the case where the speckle data is less than the first threshold, the signal generation section repeatedly performs control in a manner that the output from the laser light source is increased or decreased by a predetermined amount until the speckle data becomes the first threshold or more; and in the case where an amount of increase or an amount of decrease in the output from the laser light source exceeds a second threshold, the signal generation section generates an error signal.

This makes it possible to detect presence or absence of abnormality in the laser light source.

The speckle data may be configured to include speckle contrast, and the signal generation section may be configured to generate the control signal on the basis of the speckle contrast.

Alternatively, the image signal may be configured to include a plurality of pixel signals, each of which includes luminance information, the speckle data may be configured to include a difference between maximum luminance and minimum luminance, and the signal generation section may be configured to generate the control signal on the basis of the difference between the maximum luminance and the minimum luminance.

A control system according to an aspect of the present technology includes an illumination section, a standard sample for calibration, an image capturing section, and a control apparatus.

The illumination section includes a laser light source that emits laser light to an observation target, and a laser driver that adjusts output from the laser light source.

The standard sample is configured to be capable of being disposed at a position irradiated with the laser light.

The image capturing section acquires images of the observation target and the standard sample that have been irradiated with the laser light.

The control apparatus includes a signal generation section.

The signal generation section generates speckle data from each of pixel signals constituting the image of the standard sample, and generates a control signal for controlling the laser driver on the basis of the generated speckle data.

The control system may further include a display section.

The control apparatus further includes a display control section that causes the display section to display a speckle contrast image of the observation target.

The standard sample is typically a light diffusion optical element.

The standard sample may be a diffuser plate or may be a surgical drape.

The control system may further include a support portion that supports the standard sample. The support portion is configured to selectively switch between a first state where the standard sample is disposed in an imaging region of an imaging section and a second state where the standard sample is disposed outside the imaging region of the imaging section.

The imaging section may include a first camera that images the observation target, and a second camera that images the standard sample.

The control system may be configured as an endoscope or a microscope.

A control method according to an aspect of the present technology is a control method that is executed by a computer system. The control method includes generating speckle data on the basis of an image signal of a subject imaged by using laser light as illumination.

A control signal for controlling output from a laser light source that emits the laser light is generated on the basis of the generated speckle data.

A program according to an aspect of the present technology causes a computer system to execute:

a step of generating speckle data on the basis of an image signal of a subject imaged by using laser light as illumination; and

a step of generating a control signal for controlling output from a laser light source that emits the laser light on the basis of the generated speckle data.

### Advantageous Effects of Invention

As described above, according to the present technology, it is possible to observe a living tissue or the like with high accuracy.

Note that, the effects described herein are not necessarily limited and may be any of the effects described in the present disclosure.

### BRIEF DESCRIPTION OF DRAWINGS

FIG. 1 is a block diagram schematically illustrating a typical system configuration of a speckle blood flow imaging apparatus.

FIG. 2 is examples of images obtained by imaging blood vessels of a brain. A illustrates a bright field image, B illustrates an ICG image, and C illustrates a speckle contrast image.

FIG. 3 is a schematic configuration diagram illustrating a control system according to a first embodiment of the present technology.

FIG. 4 is a functional block diagram of the control system.

FIG. 5 is an example of a speckle image imported after emission of laser light to a model that imitates a brain.

FIG. 6 is an explanatory diagram of calculation units of speckle contrast.

FIG. 7 is a diagram illustrating an example of a speckle contrast image.

FIG. 8 illustrates experimental results obtained when speckle contrast is measured by using three LDs of a same type.

FIG. 9 illustrates a correspondence between speckle images and LD spectra.

FIG. 10 is an example of a relation between LD electric current and speckle contrast.

FIG. 11 is a flowchart illustrating basic operation of the control system.

FIG. 12 is a flowchart illustrating an example of a process procedure of a control apparatus in the control system.

FIG. 13 is a schematic configuration diagram illustrating a control system according to a second embodiment of the present technology.

FIG. 14 is a schematic configuration diagram illustrating a control system according to a third embodiment of the present technology.

FIG. 15 is a schematic configuration diagram illustrating a control system according to a fourth embodiment of the present technology.

FIG. 16 is a schematic plan view of a configuration example of a support portion in the control system.

FIG. 17 is a schematic configuration diagram illustrating a control system according to a fifth embodiment of the present technology.

FIG. 18 is a schematic configuration diagram illustrating the control system according to the fifth embodiment of the present technology.

FIG. 19 is a schematic diagram illustrating an example of an image acquired in the control system.

FIG. 20 illustrates an example in which a spectrum width of an LD varies depending on temperatures.

### MODE(S) FOR CARRYING OUT THE INVENTION

Hereinafter, embodiments of the present technology will be described with reference to the drawings.

### First Embodiment

A control system according to this embodiment is applied to observation of a living tissue or the like. Here, for example, an example of application to observation of a blood flow of a brain will be described. This can be used during a brain surgery.

[Overview of Technology]

In general, as a method of observing a blood flow, methods using ultrasonic Doppler apparatuses, ICG fluorescence methods, and the like have been in practical use. In the method using an ultrasonic Doppler apparatus, a probe that generates ultrasound is brought into contact with a blood vessel, and a state of a blood flow is recognized by using a Doppler effect between the ultrasound and the blood flow. In the ICG fluorescence method, an indocyanine green (ICG) fluorophore is injected into veins, and a blood flow is observed through luminescence caused by reaction between near-infrared light and the ICG flowing in the blood vessels.

On the other hand, speckle blood flow imaging apparatuses have also been proposed. The speckle blood flow imaging apparatus emits laser light to blood vessels and displays a blood flow by using a speckle image. The speckle image is an image obtained by emitting laser light having a specific wavelength with high interference. The wavelength may be red, blue, green, infrared, or ultraviolet. The infrared is desirable for observation of a blood flow.

The speckle blood flow imaging apparatus emits laser light with high interference to scattering material in a fluid (such as blood flowing in blood vessels), and uses a phenomenon of reduction in visibility of speckles due to their flow, is the speckles being interfering light generated using the scattering material. For example, with regard to the speckles, speckle contrast is defined as a difference between a bright part and a dark part. In other words, the speckle blood flow imaging apparatus is an apparatus that observes presence or absence of a flow (presence or absence of a blood flow) by using a phenomenon in which the speckle contrast becomes large when blood is not flowing and a phenomenon in which the speckle contrast becomes small when the blood is flowing.

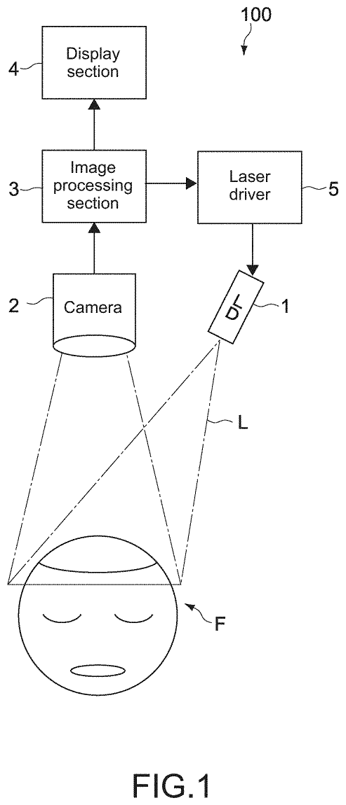

FIG. 1 is a block diagram schematically illustrating a typical system configuration of the speckle blood flow imaging apparatus. A speckle blood flow imaging apparatus 100 illustrated in FIG. 1 includes: a light source 1 that emits laser light L to a subject F as illumination light; a camera 2 that acquires a speckle image of the subject F; an image processing section 3 that processes the speckle image and computes speckle contrast; a display section 4 capable of displaying a speckle contrast image; and a laser driver 5 that controls output from an illumination section 1 on the basis of the speckle contrast.

The speckle contrast image is an image obtained by subjecting the speckle image to a signal process based on speckle contrast calculation and an image process as an image suitable for the display section 4. The combination of the signal process and the image process is referred to as an image process. Note that, a method of calculating the speckle contrast will be described later.

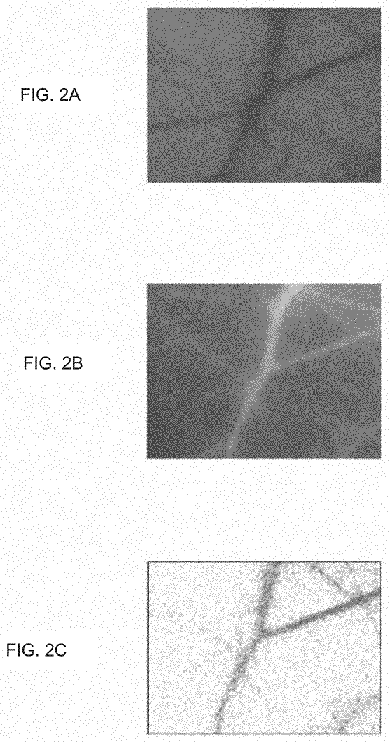

FIG. 2 illustrates an example of the speckle contrast image obtained by imaging blood vessels of a brain in comparison with a bright field image and an ICG image. In FIG. 2, A illustrates the bright field image, B illustrates the ICG image, and C illustrates the speckle contrast image. Note that, for example, the bright field image is an image obtained by emitting white light from a mercury lamp, a xenon lamp, or the like, laser light including red, blue, and green (the laser light may further include infrared and ultraviolet), or white light from an LED.

As described above, since the speckle blood flow imaging apparatus uses a situation in which a flow (movement) of scattering material makes it difficult to see the speckle image generated through scattering, original coherence (interference) of the laser light source is important. In general, coherence of laser light increases as a spectrum width gets narrower. Therefore, a laser with a narrow spectrum width is necessary for the speckle blood flow imaging apparatus. Thanks to progress in semiconductor laser technologies, inexpensive Fabry-Perot semiconductor lasers with narrow spectrum width have been in practical use. Such a Fabry-Perot semiconductor laser achieves relatively high light output. This is favorable for the light source of the speckle blood flow imaging apparatus.

However, as described later, such a semiconductor laser has a problem of widening/shrinking of the spectrum width that occurs when an amount of electrical current is changed to adjusting the light output. In addition, in general, a measuring instrument dedicated to measurement of spectrum widths is necessary to recognize a state of the laser. However, installation of such a dedicated measuring instrument into the apparatus may cause a problem of increase in the entire size of the system and complication of its configuration.

Therefore, a purpose of this embodiment is to provide a control system that makes it possible to recognize a state of a light source in the speckle blood flow imaging apparatus using a laser light source through a simple method, and stably acquire a highly accurate speckle contrast image.

Next, details thereof will be described.

[Control System]

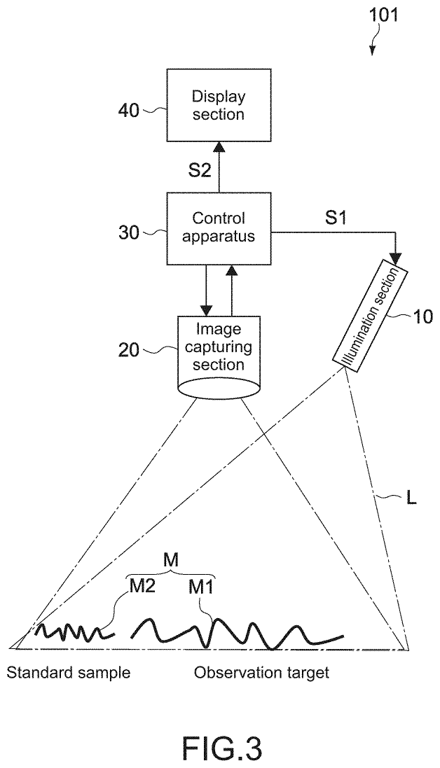

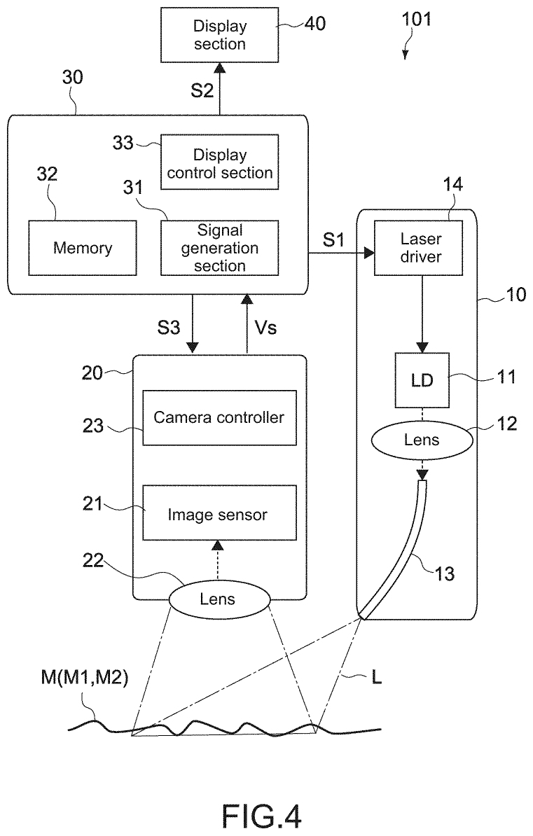

FIG. 3 is a schematic configuration diagram illustrating a control system 101 according to the first embodiment of the present technology. FIG. 4 is a functional block diagram of the control system 101 illustrated in FIG. 3.

The control system 101 includes an illumination section 10, an image capturing section 20, and a control apparatus 30. The control system 101 according to this embodiment further includes a display section 40.

The illumination section 10 emits laser light L to a subject M. The laser light L is used as illumination when a speckle image of the subject M is acquired. As illustrated in FIG. 4, the illumination section 10 includes a laser light source 11, an illumination lens 12, an optical fiber 13, and a laser driver 14. The illumination section 10 may further include a temperature adjustment portion (not illustrated) that maintains the laser light source 11 at a predetermined temperature.

The laser light source 11 generates the laser light L to be emitted to the subject M. The laser light source 11 includes a laser diode (LD). For example, the laser light source 11 includes the Fabry-Perot semiconductor laser. The wavelength of the laser light L emitted from the laser light source 11 is not specifically limited. The wavelength may be a visible light wavelength such as red, blue, or green, or may be a wavelength in an infrared or ultraviolet region. It is possible to adopt an appropriate laser wavelength that makes it possible to obtain a desired speckle image of the subject M. A near-infrared laser light source is favorable as the laser light source 1 in the case where a purpose is to observe a blood flow in blood vessels like this embodiment.

The illumination lens 12 collects laser light emitted from the laser light source 11 at an input end of the optical fiber 13. The optical fiber 13 transmits the laser light L incident on the input end to an output end, and emits the laser light L from the output end to the subject M. Usage of the optical fiber 13 makes it possible to arbitrarily adjust an emission position and an emission direction of the laser light L1. This makes it easier to handle the illumination in comparison with a case where the laser light source 11 directly emits the laser light L to the subject M.

Note that, if necessary, the optical fiber 13 may be omitted. In this case, the illumination lens 12 is configured to adjust an emission range of the laser light L in a manner that the laser light source 11 emits the laser light L to a predetermined region of the subject M.

The laser driver 14 adjusts output from the laser light source 11. The laser driver 14 typically adjusts the output from the laser light source 11 by adjusting a driving electric current of the laser light source 11. An adjustable range of the output from the laser light source 11 is appropriately set in accordance with a type or a specification of the laser light source 11. For example, the adjustable range is a range from 200 mW to 400 mW.

As described later, the laser driver 14 is driven on the basis of a control signal S1 (see FIG. 4) output from the control apparatus 30.

The subject M includes an observation target M1 and a standard sample M2 for calibration. The observation target M1 includes blood vessels of a brain or the like of a patient. As the standard sample M2, an optical element is typically used. The optical element includes a uniform medium having a light diffusion property that makes it possible to reflect the laser light L toward the image capturing section 20. As described later, the standard sample M2 is referred to when output from the laser light source 11 is evaluated.

In this embodiment, a light reflective diffuser plate is used as the standard sample M2. However, as described later, another member such as a surgical drape may also serve as the standard sample M2. The standard sample M2 is disposed at a position capable of being irradiated with the laser light L. Typically, the standard sample M2 is disposed near the observation target M1. The standard sample M2 is constantly disposed in an imaging region. However, as described later, the standard sample M2 may be configured to be capable of being disposed in the imaging region at any timing.

The image capturing section 20 images the observation target M1 and the standard sample M2 that are irradiated with the laser light L, and acquires an image (speckle image) of the subject M. The speckle image of the subject M includes a speckle image of the observation target M1 and a speckle image of the standard sample M2. Among them, the speckle image of the standard sample M2 may also be referred to as a standard speckle image.

As illustrated in FIG. 4, the image capturing section 20 includes an image sensor 21, a lens system 22, and a camera controller 23.

The image sensor captures an image of the subject M irradiated with the laser light L, and outputs the image to the control apparatus 30 as an image signal Vs (see FIG. 4). As an image capturing element included in the image sensor 21, it is possible to use a charge coupled device (CCD) sensor, a complementary metal oxide semiconductor (CMOS) sensor, or the like, for example. Of course, it is possible to use another type of image capturing element.

The lens system 22 forms a reflection image of the laser light L reflected from the subject M, on the image capturing element of the image sensor 21. The lens system 22 typically includes a plurality of optical lenses and a diaphragm, and is configured to be movable in an optical axis direction via the camera controller 23.

The camera controller 23 drives the image sensor 21 and performs control to import an image from the image sensor 21 on the basis of a control instruction S3 (see FIG. 4) from the control apparatus 30. The camera controller 23 is configured to be capable of controlling imaging parameters of the image capturing section 20. The imaging parameters related to the imaging include any parameter related to imaging of the subject M. The imaging parameters include any parameters such as exposure time, gain of the image capturing element, a focal length, an angle of view, and an f-number.

Note that, the camera controller 23 may be configured as a part of the control apparatus 30.

In this embodiment, a single camera (image sensor 21) images the observation target M1 and the standard sample M2 at a same time. Therefore, each of pixel signals constituting the image signal Vs of the subject M includes pixel signals related to the observation target M2 and pixel signals related to the standard sample M2.

Note that, as described later, the observation target M1 and the standard sample M2 may be independently imaged by a single camera (at different times), or may be simultaneously imaged by two cameras (at a same time).

[Control Apparatus]

Next, the control apparatus 30 will be described.

The control apparatus 30 includes hardware that is necessary for configuring a computer such as a CPU, ROM, RAM, and an HDD. The control method according to the present technology is executed when the CPU loads a program into the RAM and executes the program. The program relates to the present technology and is recorded in the ROM or the like in advance. For example, the control apparatus 30 can be implemented by any computer such as a personal computer (PC).

The specific configuration of the control apparatus 30 is not limited. For example, it is possible to use a field programmable gate array (FPGA), an image processing integrated circuit (IC), or another device such as an application specific integrated circuit (ASIC).

The control apparatus 30 controls the illumination section 10, the image capturing section 20, and the display section 40. As illustrated in FIG. 4, in this embodiment, a signal generation section 31 and a display control section 33 are configured as functional blocks when the CPU executes a predetermined program. In addition, memory 32 is implemented by ROM or the like of the control apparatus 30. Of course, it is also possible to use dedicated hardware such as an integrated circuit (IC) to implement the respective blocks. The program is installed in the control apparatus 30 via various kinds of recording media, for example. Alternatively, it is also possible to install the program via the Internet.

The signal generation section 31 generates speckle data on the basis of an image signal of the subject M imaged by using the laser light L as illumination, and generates the control signal S1 for controlling output from the laser light source 11 that emits the laser light L on the basis of the generated speckle data.

In this embodiment, the signal generation section 31 generates speckle data from each of pixel signals (in other words, standard speckle image) constituting the image of the standard sample M2, and generates the control signal S1 for controlling the laser driver 14 on the basis of the generated speckle data. The speckle data includes speckle contrast. The signal generation section 31 generates the control signal S1 on the basis of the speckle contrast.

The memory 32 includes frame memory capable of storing the image signal Vs input from the image capturing section 30. The memory 32 includes non-volatile memory that fixedly stores arithmetic parameters necessary for generating the speckle contrast (speckle data) of the signal generation section 31.

The display control section 33 generates speckle contrast images of the observation target M1 and the standard sample M2 on the basis of the speckle data of the subject M generated by the signal generation section 31, and outputs a display signal S2 (see FIG. 4) to the display section 40. The display signal S2 causes the display section 40 to display the generated speckle contrast images. For example, the display control section 33 generates 60 speckle contrast images (60 Hz) per second.

As described later, the display control section 33 is also configured to be capable of causing the display section 40 to display completion information of adjustment of output from the laser light source 11, error information of the laser light source 11, or the like in addition to the speckle contrast image of the subject M.

Note that, the display control section 33 may be configured to be capable of causing the display section 40 to display a speckle image, a bright field image, an ICG image, or the like of the subject M in addition to the speckle contrast image of the subject M. It is possible to selectively display the plurality of such images at a same time, or it is possible to alternately display each of such images. In the case where an illumination section (not illustrated) for acquiring the bright field images is installed in addition to the illumination section 10, the control apparatus 30 may be configured to be capable of switching illumination of these illumination sections in chronological order (in a field sequential manner).

The display section 40 is not specifically limited. The display section 40 may be a monitor apparatus such as a liquid crystal display (LCD) or an organic electro-luminescence display, a viewer built in an eyepiece for an endoscope or a microscope.

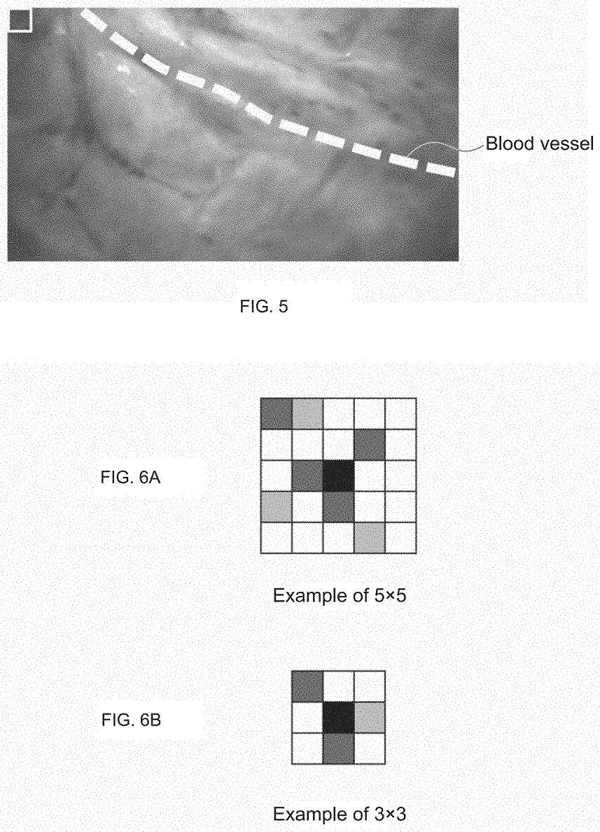

FIG. 5 is an example of a speckle image imported after emission of laser light to a model that imitates a brain. With reference to FIG. 5, a dotted line represents a hollow tube that imitates a blood vessel in which a fluid flows. In the hollow tube, a fluid including scattering material that imitates blood can flow. In FIG. 5, a region surrounded by a white square on an upper left side represents a unit of image process, for example.

FIG. 6 is an explanatory diagram of calculation units of speckle contrast. For example, the signal generation section 32 generates the speckle contrast as described below.

For example, as illustrated in FIG. 6A, when 5×5 pixels are used as a unit, the speckle contrast is defined by the following equation.

K=σ/(I) (1)

where K represents (a gradation value) of brightness of the center, σ represents a standard deviation of gradation values of peripheral pixels, and (I) represents an average value. When this equation is sequentially applied to the units of pixels in a whole screen, it is possible to obtain the speckle contrast image.

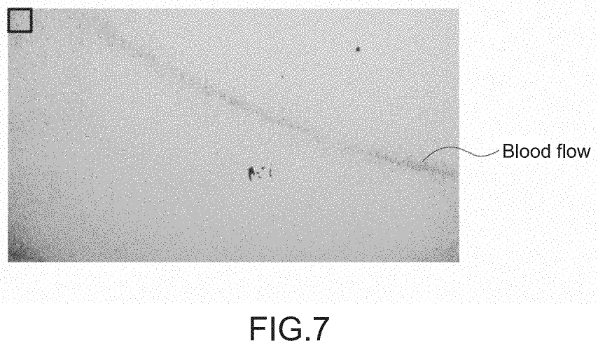

As illustrated in FIG. 6B, the unit of pixels used for the arithmetic operation of the speckle contrast may be 3×3 pixels, or it is possible to use another unit. In the case of a still state, the speckles can be seen clearly, and dispersion of the pixel values increases. Therefore, the standard deviation increases, and the value of K increases. In the case where the fluid is flowing, speckles are cluttered (speckles cannot be seen), and dispersion of the pixel values decreases. Therefore, the standard deviation decreases, and the value of K also decreases. Accordingly, the K value decreases (the image gets darker) in the case where the scattering material is moving in the blood vessels like when the blood is flowing. FIG. 7 illustrates an example of the speckle contrast image. In FIG. 7, a black linear part represents a blood flow.

As described above, the speckle contrast can be calculated only from a horizontal and vertical size of the image in the whole screen. An image obtained through this calculation is the speckle contrast image. However, it is difficult to determine which state the obtained image is in, from a normal image. Therefore, to easily obtain the speckle image, it is desirable to emit a roughly uniform amount of laser light to the standard sample. In addition, a diffuser plate is used as the standard sample. A reflective diffuser plate is especially favorable for such a purpose.

In addition, with regard to the speckle image (standard speckle image) obtained by emitting the laser light to the standard sample, sometimes values of (K values) of speckle contrast varies (increases/decreases) partially. Therefore, it is favorable to decide a representative value for comparative evaluation of the states. Various kinds of methods are considered to be used for deciding the representative value. For example, it is possible to use an average value of values of speckle contrast (speckle contrast values) of the whole screen as the speckle contrast, or it is possible to use an average value of speckle contrast values in a uniformly irradiated region as the representative value.

However, the size of the speckle contrast depends on the state of the light source, the optical system, the diaphragm of the lens, the subject, and the like. In particular, an oscillation spectrum of the laser light source easily varies. Therefore, the obtained speckle contrast is easily affected by the variation in the oscillation spectrum of the laser light source.

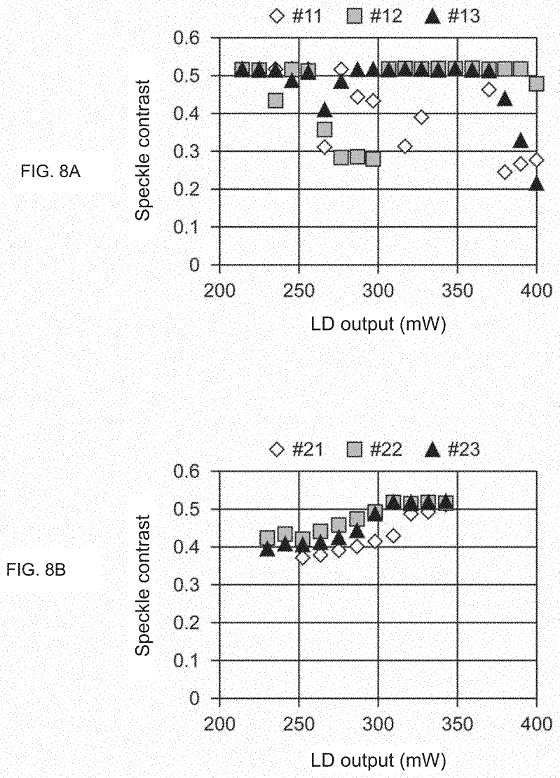

For example, FIG. 8A and FIG. 8B illustrate experimental results obtained when speckle contrast is measured by using three LDs of a same type. FIG. 8A illustrates an example of measurement using three LDs (#11, #12, and #13) manufactured by a same manufacturer. FIG. 8B illustrates an example of measurement using three LDs (#21, #22, and #23) manufactured by different manufacturers. As illustrated in FIG. 8A and FIG. 8B, the speckle contrast varies depending on change in LD output. In particular, in FIG. 8A, the speckle contrast decreases at random. It is understood that there are individual differences even when the LDs of the same type are used.

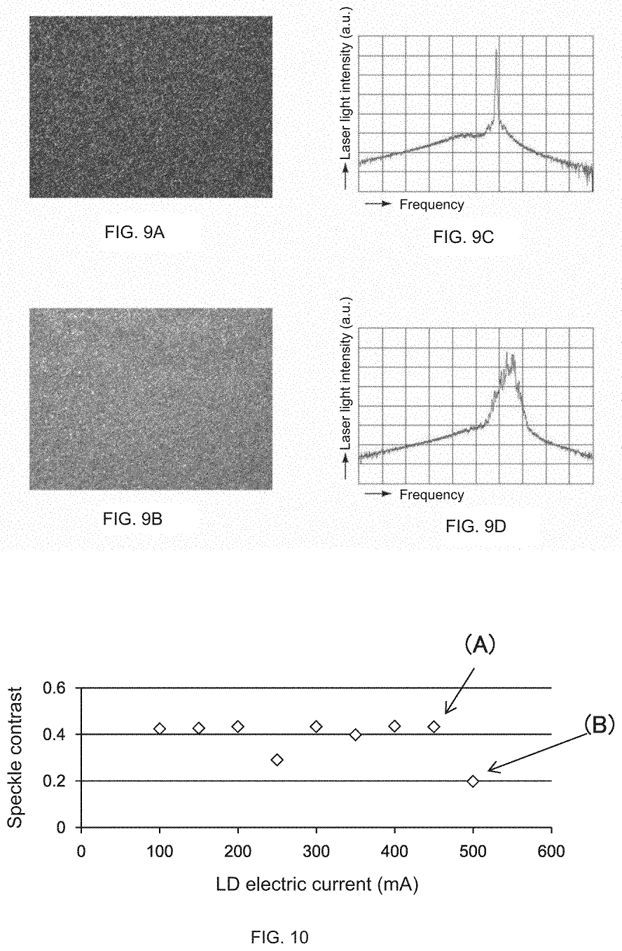

FIG. 9 illustrates a correspondence between speckle images and LD spectra. FIG. 10 is an example of a relation between LD electric current and speckle contrast. FIG. 9C corresponds to the LD spectrum associated with the speckle image in FIG. 9A. FIG. 9D corresponds to the LD spectrum associated with the speckle image in FIG. 9B. (A) in FIG. 10 represents the speckle contrast of the speckle image in FIG. 9A, and (B) in FIG. 10 represents the speckle contrast of the speckle image in FIG. 9B. High speckle contrast is obtained in the case where the spectrum width is narrow, and low speckle contrast is obtained in the case where the spectrum width is wide.

As described above, the spectrum width of the semiconductor laser used as the laser light source varies depending on the magnitude of the output (driving electric current). Therefore, sometimes it is impossible to appropriately acquire a speckle image of a subject with desired speckle contrast even under the same oscillation conditions.

Therefore, according to this embodiment, it is evaluated whether it is possible to obtain a speckle image with desired speckle contrast from the laser light emitted from the illumination section 10 before the image of the observation target M1 is observed. In the case where the desired speckle contrast is not obtained, output from the laser light source 11 is adjusted to obtain the desired speckle contrast. As described above, it is possible to stably observe the image with high accuracy by calibrating the output from the laser light source before the image is actually observed.

[Operation of Control System]

Next, details of the control apparatus 30 and typical operation of the control system 101 will be described.

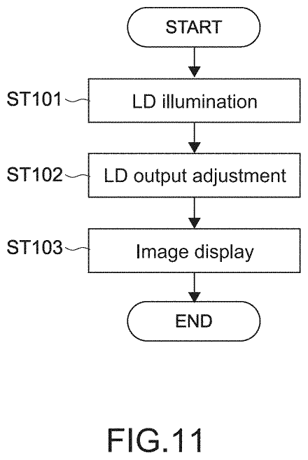

FIG. 11 is a flowchart illustrating basic operation of the control system 101. The control system 101 includes a step (Step 101) of emitting the laser light L, a step (Step 102) of adjusting output of the laser light L, and a step (Step 103) of displaying an image. In the step of adjusting the output of the laser light L, speckle contrast is evaluated on the basis of a speckle image of the subject M imaged by using the laser light L as illumination, and the output of the laser light L is adjusted in the case where the speckle contrast is less than a predetermined threshold. In the step of displaying an image, the display section 40 displays a result of adjusting the output from the laser light L and the speckle contrast image of the subject.

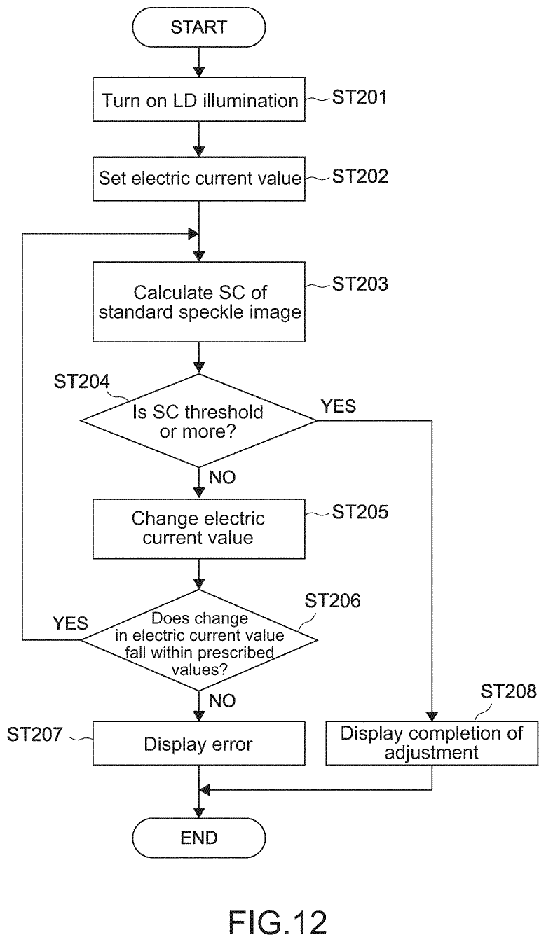

FIG. 12 is a flowchart illustrating an example of a process procedure of the control apparatus 30.

The control apparatus 30 outputs an instruction to turn on the laser light source 11 to the laser driver 14 as the control signal S1 (Step 201). Next, a driving electric current of the laser light L is adjusted to a set electric current value (such as 300 mA), and the laser light L is emitted to the subject M (Step 202). Such processes correspond to the process in Step 101 illustrated in FIG. 11.

Next, the control apparatus 30 (the signal generation section 32) acquires a speckle image of the subject M irradiated with the laser light L via the image capturing section 20, and computes speckle data (in this example, speckle contrast (SC)) of the speckle image on the basis of its image signal Vs through the above-described arithmetic method (Step 203). In this embodiment, speckle contrast is computed on the basis of the speckle image (standard speckle image) of the standard sample M2 (diffuser plate) in the speckle image of the subject M.

The signal generation section 32 determines whether or not the speckle contrast of the standard sample M2 is a predetermined threshold (hereinafter, referred to as a first threshold) or more. In the case where the speckle contrast of the standard sample M2 is the first threshold or more, the signal generation section 32 causes the display section 40 to display indication of completion of the adjustment (Step 208) The display control is performed when the display control section 33 outputs the control signal S2 to the display section 40.

On the other hand, in the case where the speckle contrast is less than the first threshold, the signal generation section 32 generates the control signal S1 for increasing or decreasing the output from the laser light source 11 in a manner that the speckle contrast becomes the first threshold or more. Subsequently, the signal generation section 32 outputs the control signal S1 to the laser driver 14 (Step 205). This makes it possible to control the laser light source 11 in a manner that a high-definition observation image with desired speckle contrast is obtained from its output.

The first threshold is not specifically limited. The first threshold can be appropriately set depending on a content or the like of the observation target M1. For example, the first threshold is set to 0.4 in the case of blood flow observation.

In the case where the speckle data is less than the first threshold, the signal generation section 32 repeatedly performs control in a manner that the output from the laser light source 11 is increased or decreased by a predetermined amount until the speckle data becomes the first threshold or more (Step 203 to Step 206). As illustrated in FIG. 8 and FIG. 10, this is because sometimes the speckle contrast is improved through delicate adjustment of the driving electric current value.

For example, the signal generation section 32 delicately adjusts the electric current value of the laser light source 11 to a value (290 mA) obtained by subtracting 10 mA from the set electric current value (300 mA), and determines whether or not speckle contrast of the standard speckle image after the delicate adjustment is the first threshold or more. Next, in the case where the speckle contrast is the first threshold or more, the display section 40 displays indication of completion of the adjustment of the laser output (Step 208).

On the other hand, in the case where the speckle contrast is less than the first threshold even after the above-described delicate adjustment, the signal generation section 32 delicately adjusts the electric current value of the laser light source 11 to a value (310 mA) obtained by adding 10 mA to the set electric current value, and evaluates the speckle contrast again. Subsequently, the signal generation section 32 sequentially changes the electric current value of the laser light source 11 to a value (280 mA) obtained by subtracting 20 mA from the set electric current value, a value (320 mA) obtained by adding 20 mA to the set electric current value, a value (270 mA) obtained by subtracting 30 mA from the set electric current value, and a value (330 mA) obtained by adding 30 mA to the set electric current value until the speckle contrast of the first threshold or more is obtained. Note that, the adjustable range of the driving electric current is not limited to 10 mA. The adjustable range may be set to an appropriate value such as 5 mA.

On the other hand, if the adjustable range of the driving electric current is away from the set value too much, the image gets too bright or too dark, and it becomes impossible to obtain desired brightness. Therefore, in the case where an amount of increase or an amount of decrease in the output from the laser light source exceeds a second threshold (such as ±30 mA), the signal generation section 32 generates an error signal indicating abnormality of the laser light source 11, and causes the display section 40 to display indication of the abnormality (Step 207). This makes it possible to detect presence or absence of abnormality in the laser light source 11. Therefore, it is possible to provide a user with an announcement that prompts the user to inspect the illumination section 10 or replace the laser light source 11 with a new one, or the like.

As described above, in this embodiment, the control apparatus 30 is configured to control the oscillation spectrum of the laser light source 11 on the basis of speckle data computed from the image signal of the subject M. This makes it possible to suppress variation in the oscillation spectrum of the laser light source 11 and observe the blood flow in the blood vessels of the brain or the like with high accuracy.

In particular, in this embodiment, the oscillation spectrum of the laser light source 11 is calibrated by using the speckle image (standard speckle image) of the standard sample M2. This makes it possible to detect the oscillation spectrum of the laser light source and its variation with high accuracy.

In addition, in this embodiment, the standard sample M2 is disposed in the same surgical field (imaging region) as the observation target M1. This makes it possible to acquire the standard speckle image under the same illumination condition as the illumination condition of the observation target M1. This makes it possible to enhance calibration accuracy of output from the laser light source 11.

In addition, in this embodiment, the speckle image of the subject M includes the standard sample M2. This makes it possible to calibrate the output from the laser light source 11 easily and quickly not only before a surgery but also at any timing during the surgery such as after adjustment of an angle of view of the image capturing section 2.

In addition, in this embodiment, it is possible to easily recognize the state of the laser light in real time on the basis of the speckle contrast of the image without using the measuring instrument dedicated to measurement of the spectrum width of the laser light. This makes it possible to prevent the whole system from getting larger or getting complex.

### Second Embodiment

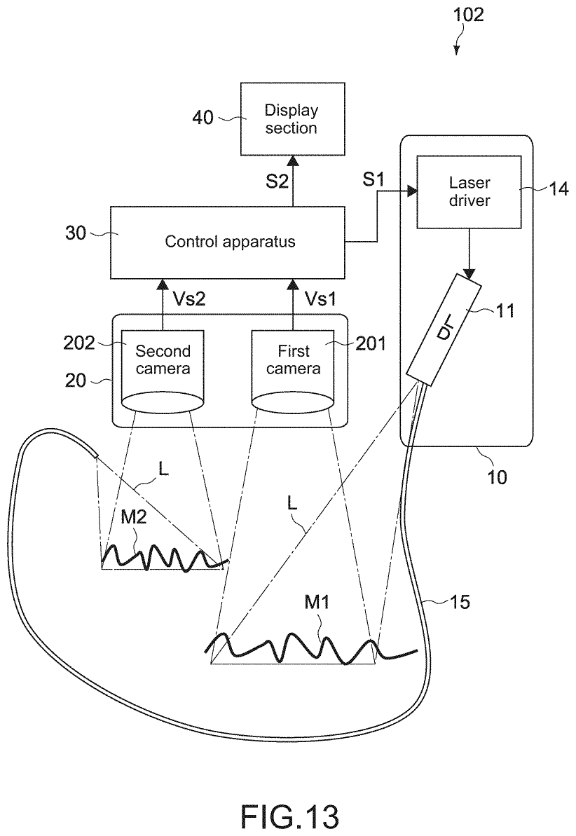

FIG. 13 is a schematic configuration diagram illustrating a control system 102 according to a second embodiment of the present technology. Hereinafter, structures different from those in the first embodiment will be mainly described. The structures that are similar to those in the first embodiment will be denoted by the same reference signs as the first embodiment, and description thereof will be omitted or simplified.

In the control system 102 according to this embodiment, the image capturing section 20 includes: a first camera 210 that images the observation target M1 serving as a subject; and a second camera 202 that images the standard sample M2. The first camera 201 acquires an image (speckle image) of the observation target M1, and outputs the image to the control apparatus 30 as a first image signal Vs1. The second camera 202 acquires an image (standard speckle image) of the standard sample M2, and outputs the image to the control apparatus 30 as a second image signal Vs2.

The illumination section 10 includes: the laser light source 11 that emits laser light L1 to the observation target M1; and an optical fiber 15 that emits laser light L to the standard sample M2. For example, the optical fiber 15 is connected to an output end of the laser light source 11, and the optical fiber 15 includes a branching optical fiber that divides the laser light L emitted from the laser light source 11 and emits a beam of the laser light L to the standard sample M2. This makes it possible to emit the laser light L from the same laser light source 11 to the observation target M1 and the standard sample M2 at the same time.

The control apparatus 30 computes speckle contrast on the basis of the second image signal Vs2 output from the second camera 202. In a way similar to the first embodiment, in the case where the computed value is less than the first threshold, the control apparatus 30 generates the control signal S1 for controlling the output from the laser light source 11 in a manner that the speckle contrast becomes the first threshold or more. The control apparatus 30 generates a speckle contrast image of the observation target M1 on the basis of the first image signal Vs1 output from the first camera 201, and causes the display section 40 to display the generated speckle contrast image.

The control system 102 configured as described above according to this embodiment can achieve operation/effects similar to the first embodiment. According to this embodiment, it is possible to obtain a standard speckle image that is necessary for adjusting the output from the laser light source 11 even in the case where the standard sample M2 is disposed at a position relatively distant from the observation target M1.

In addition, it is possible to individually optimize respective illumination conditions of the laser light L with regard to the observation target M1 and the standard sample M2. In addition, it is possible to individually optimize respective imaging conditions of the cameras 201 and 202 because the observation target M1 and the standard sample M2 can be respectively imaged by the different cameras 201 and 202. As a result, it is possible to easily acquire respective high-definition speckle images of the observation target M1 and the standard sample M2.

### Third Embodiment

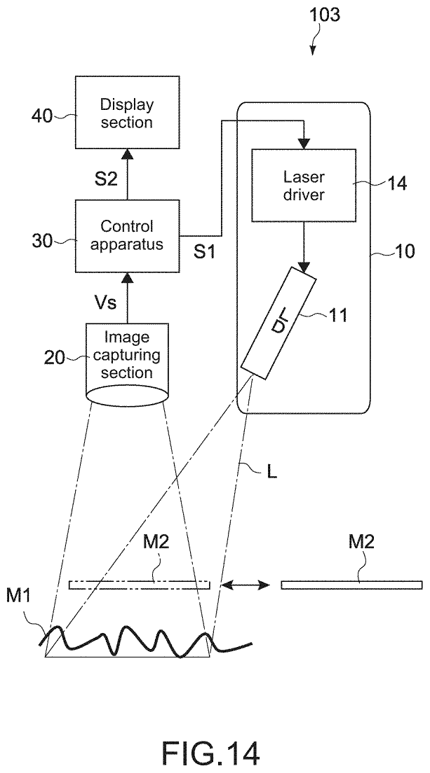

FIG. 14 is a schematic configuration diagram illustrating a control system 103 according to a third embodiment of the present technology. Hereinafter, structures different from those in the first embodiment will be mainly described. The structures that are similar to those in the first embodiment will be denoted by the same reference signs as the first embodiment, and description thereof will be omitted or simplified.

The control system 103 according to this embodiment is different from the first embodiment in that the standard sample M2 is configured to be movable relative to the observation target M1. In other words, the standard sample M2 is configured to be movable back and forth between an evacuation position indicated by a solid line in FIG. 14 and an imaging position indicated by a dashed double-dotted line in FIG. 14. The evacuation position is set to a position outside the imaging region of an imaging section 20. The imaging position is set to a position inside the imaging region of the imaging section 20. For example, the imaging position is set to a position immediately above the observation target M1.

The control system 103 according to this embodiment can achieve operation/effects similar to the first embodiment. According to the embodiment, it is possible to move the standard sample M2 to the evacuation position unless the step of adjusting the output of the laser light source 11 is performed. This makes it possible to secure a relatively wide surgical field with regard to the observation target M1.

Although not illustrated, the standard sample M2 according to this embodiment is supported by a support portion including an appropriate reciprocating movement mechanism such as an air cylinder or a linear motor. As described above, the support portion is configured to selectively switch between a first state where the standard sample M2 is disposed in the imaging region of the imaging section and a second state where the standard sample M2 is disposed outside the imaging region of the imaging section 20. The configuration of the support portion is not limited thereto. As described later, the support portion may include a rotation mechanism.

### Fourth Embodiment

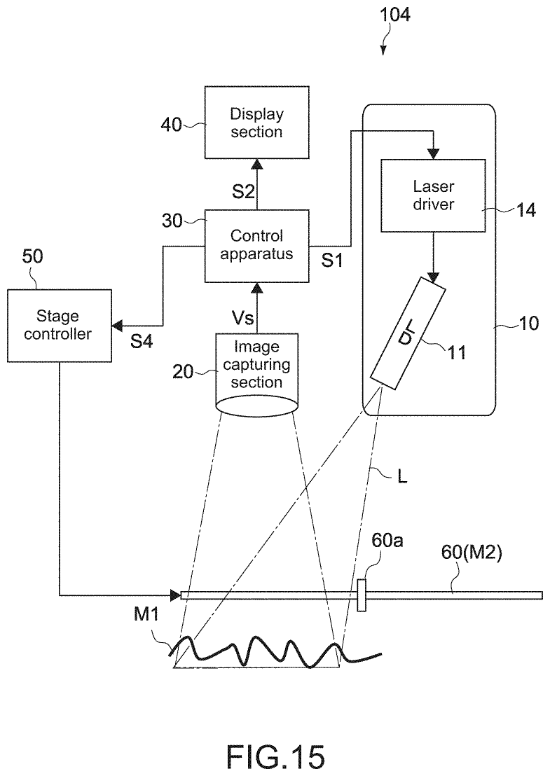

FIG. 15 is a schematic configuration diagram illustrating a control system 104 according to a fourth embodiment of the present technology. Hereinafter, structures different from those in the first embodiment will be mainly described. The structures that are similar to those in the first embodiment will be denoted by the same reference signs as the first embodiment, and description thereof will be omitted or simplified.

The control system 104 according to this embodiment is different from the control system 101 according to the first embodiment in that the standard sample M2 is configured to be movable relative to the observation target M1. The control system 104 according to this embodiment includes: a rotation stage 60 serving as the support portion that supports the standard sample M2; and a stage controller 50 that controls driving of the rotation stage 60.

The rotation stage 60 is configured to rotate the standard sample M2 around a rotation shaft 60a and selectively switch between a first state where the standard sample M2 is disposed in the imaging region of the imaging section 20 and a second state where the standard sample M2 is disposed outside the imaging region of the imaging section 20.

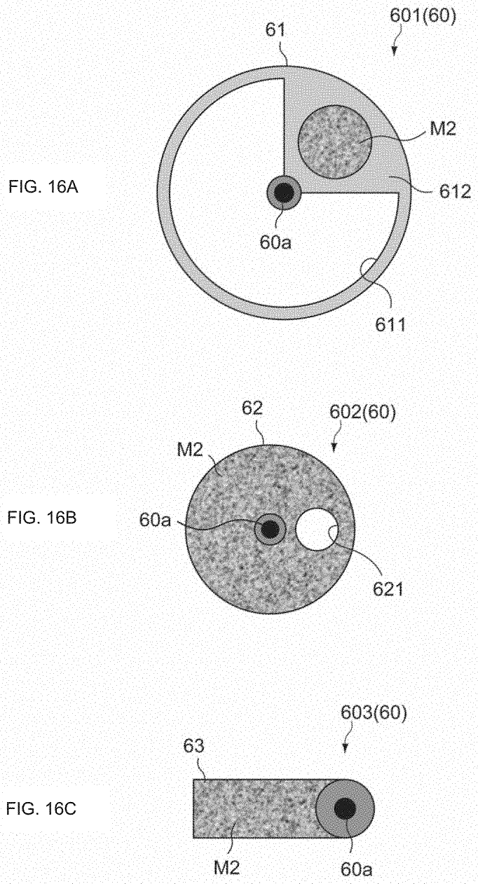

FIG. 16A to FIG. 16C are schematic plan views of configuration examples of the rotation stage 60.

A rotation stage 601 illustrated in FIG. 16A includes a circular stage main body 61 having a rotation shaft 60a at its center. The stage main body 61 has an opening 611 and a support section 612 for supporting the standard sample M2. The opening 611 has a circular sector shape, and the opening 611 makes it possible to open the image capturing region of the observation target M1 to the image capturing section 20 and allow the image capturing section 20 to image the observation target M1. The central angle of the opening 611 is not specifically limited. The central angle may be smaller or larger than the angle (approximately 270 degrees) illustrated in FIG. 16 as long as the size of the opening is enough to secure the image capturing region of the observation target M1. The support section 612 has a size capable of disposing the standard sample M2 in the image capturing region at a predetermined rotation position. The planar shape of the standard sample M2 is not limited to the circular shape illustrated in FIG. 16. The standard sample M2 may have another geometric shape such as a rectangular shape.

In the rotation stage 601, the opening 611 has a larger area than the standard sample M2. However, the rotation stage 601 is not limited thereto. As illustrated in FIG. 16B, the standard sample M2 may have a larger area than the opening.

A rotation stage 602 illustrated in FIG. 16B includes a circular stage main body 62 having a rotation shaft 60a at its center. The circular stage main body 62 supports the standard sample M2. The stage main body 62 may be the standard sample M2. The stage main body 62 has an opening 621, and the opening 621 has a size capable of securing the image capturing region of the observation target M1 for the image capturing section 20 at a predetermined rotation position. The shape of the opening 621 is not limited to a circular shape. The opening 621 may have another geometric shape such as a rectangular shape.

A rotation stage 603 illustrated in FIG. 16C includes a rectangular stage main body 63 having a rotation shaft 60a at its one end. The rectangular stage main body 63 supports the standard sample M2. The stage main body 63 may be the standard sample M2. The stage main body 63 has an appropriate size capable of being disposed in the image capturing region of the image capturing section 20 at a predetermined rotation position.

The stage controller 50 is configured to be capable of rotating the rotation stage 60 at any angle on the basis of a switch instruction S4 from the control apparatus 30. The stage controller 50 may be configured as a part of the control apparatus 30. Note that, the rotation stage 60 may be configured to be rotatable by a user with his/her hand.

The control system 104 according to this embodiment can achieve operation/effects similar to the first embodiment. According to this embodiment, it is possible to move the standard sample M2 to the evacuation position unless the step of adjusting the output of the laser light source 11 is performed. This makes it possible to secure a relatively wide surgical field with regard to the observation target M1.

### Fifth Embodiment

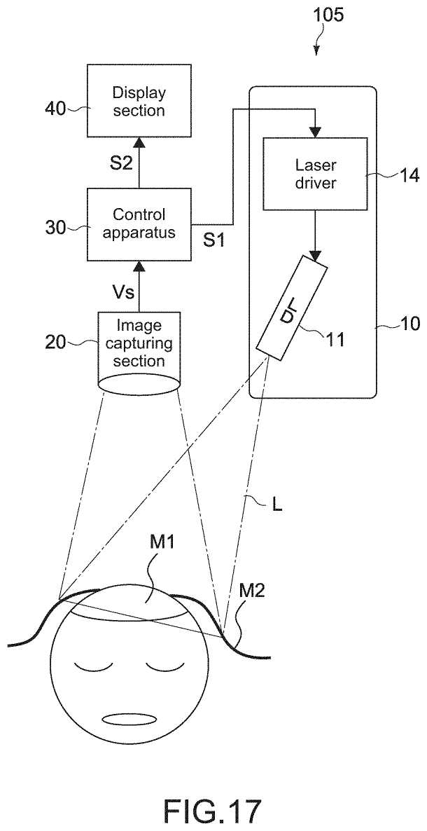

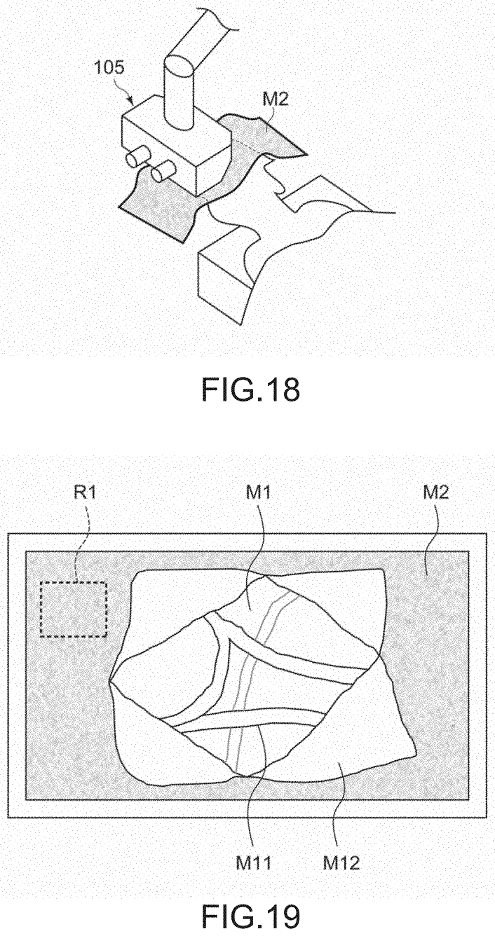

FIG. 17 and FIG. 18 are schematic configuration diagrams illustrating a control system 105 according to a fifth embodiment of the present technology. Hereinafter, structures different from those in the first embodiment will be mainly described. The structures that are similar to those in the first embodiment will be denoted by the same reference signs as the first embodiment, and description thereof will be omitted or simplified.

In this embodiment, the control system 105 is applied to a microscope for brain surgery. As the standard sample M2, a general-purpose surgical drape (cloth) from which a standard speckle image is obtained is used. The illumination section 10 emits laser light L to a head of a patient. The image capturing section 20 acquires an image (speckle image) of the head of the patient (observation target M1) and the drape (standard sample M2) around the head at a same time. FIG. 19 schematically illustrates and example of the image. In FIG. 19, M11 represents blood vessels, and M12 represents dura mater.

The control apparatus 30 computes speckle contrast of the image from the speckle image of the drape (M2). A region R1 indicated by a dashed line in FIG. 19 is a pixel indication region for calculating a speckle contrast value. The region R1 may be arbitrarily selected by the control apparatus 30, or may be selected through user operation.

The control apparatus 30 is configured in a manner that: the control apparatus 30 is capable of storing the speckle contrast value obtained at any timing (for example, in the case where the illumination section 10 is turned on at a start time in the case where the user indicates the speckle contrast calculation region R1, or the like); the control apparatus 30 is capable of comparing the obtained speckle images and the speckle contrast values in the case where an amount of illumination light is changed; and the control apparatus 30 is capable of displaying an error in the case where a gap of a predetermined value or more is observed (for example, a difference in speckle contrast is 0.1 or more). In the case where the surgical drape is used as the standard sample M2, sometimes low speckle contrast is obtained. Therefore, it is also possible to use the diffuser plate at the start time and observe variation in the speckle contrast value at any set place during the surgery.

### Another Embodiment

The present technology is not limited to the above-described embodiments. Various other embodiments are possible.

For example, in the above-described embodiments, output of laser light is calibrated on the basis of speckle contrast generated by using a speckle image (standard speckle image) of the standard sample M2. Alternatively, it is possible to calibrate output of laser light without using the standard sample M2. For example, it is possible to calibrate the output of the laser light by using the speckle image of the observation target M1. In this case, a region of the observation target M1 in which speckle contrast values are constant may be used as a representative value.

In addition, in the above-described embodiments, speckle contrast is computed as speckle data. However, the present technology is not limited thereto. For example, the speckle data may be generated on the basis of a difference between maximum luminance and minimum luminance of pixel signals in a unit cell. Specifically, with regard to the unit cell (such as 5×5 pixel array) included in a speckle image, a value ((Gmax−Gmin)/(I)) obtained by dividing the difference between maximum luminance and minimum luminance by an average value may be used. In addition, it is possible to generate a control signal for adjusting the output from the laser light source on the basis of the speckle data generated through such an arithmetic method.

In addition, in the above-described embodiments, the speckle contrast of the standard sample M2 is evaluated at a time of calibrating the output from the laser light source 11. However, the present technology is applicable to temperature management of the laser light source 11.

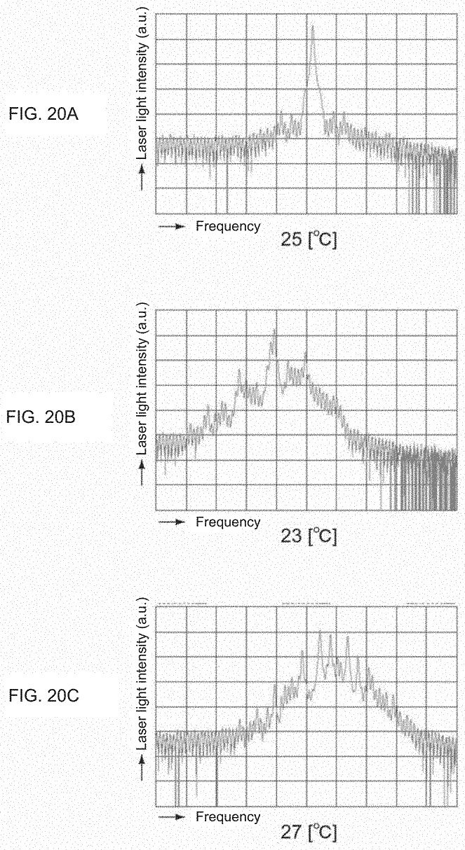

For example, FIG. 20A to FIG. 20C illustrate examples in which a spectrum width of an LD varies depending on temperatures. FIG. 20A illustrates a spectrum width obtained at 25° C., FIG. 20B illustrates a spectrum width obtained at 23° C., and FIG. 20C illustrates a spectrum width obtained at 27° C. As illustrated in FIG. 20, the spectrum width of the LD varies depending on temperatures. Therefore, speckle contrast obtained by emitting the laser light to the standard sample also varies in each state. With reference to FIG. 20A to FIG. 20C, the state illustrated in FIG. 20A has a smaller spectrum width than the states illustrated in FIG. 20B and FIG. 20C. Therefore, the highest speckle contrast is obtained in the state illustrated in FIG. 20A.

By using the variation in the spectrum width of the LD depending on temperatures, it is possible to cause the control apparatus 30 to execute the following process. In other words, as a usual procedure, an electric current value (output) of the LD is adjusted while adjusting the LD temperature, a state of the LD is checked through calculation of speckle contrast, and a speckle image is acquired. For example, 60 images are always acquired per second (in the case of 60 Hz), and the speckle contrast is calculated. In the case where the speckle contrast images are always observed, the temperature of the LD is adjusted by a Peltier element and is always maintained at a constant temperature, such as 25° C. in a normal state. However, in the case where it becomes impossible to adjust the temperature of the LD due to some sort of trouble such as Peltier element failure, or in the case where a fan for letting heat out does not work and it becomes impossible to control the Peltier element, the speckle contrast decreases. Therefore, when such decrease in the contrast is detected, the control apparatus 30 causes the display section 40 to display an error, or the control apparatus 30 exerts a process of reducing output from the illumination section 10. This makes it possible to prevent the LD from getting overheated and breaking down.

For example, the present technology is applicable to an endoscope, an optical microscope, and the like that are used in a medical/biological fields. In other words, the control system may be configured as the endoscope or the microscope.

In this case, examples of the observation target include a living tissue such as a cell, a tissue, or an organ of a living body. When using the present technology, it is possible to observe a living tissue with high accuracy. For example, when the process illustrated in FIG. 12 is executed on the basis of a speckle image captured by the endoscope or the optical microscope, it is possible to suppress variation in output from the laser light source, and observe the blood flow in the blood vessels of the brain or the like with high accuracy.

In addition, when a computer operated by a user or the like and another computer capable of communication via a network work in conjunction with each other, the control method and the program according to the present technology are executed, and this makes it possible to configure the control system according to the present technology.

That is, the control method and the program according to the present technology can be executed not only in a computer system configured by a single computer but also in a computer system in which a plurality of computers cooperatively operates. It should be noted that in the present disclosure, the system means an aggregate of a plurality of components (apparatus, module (parts), and the like) and it does not matter whether or not all the components are housed in the same casing. Therefore, both a plurality of apparatuses housed in separate casings and connected to one another via a network, and a single apparatus having a plurality of modules housed in a single casing are regarded as systems.

The execution of the control method and the program according to the present technology by the computer system includes, for example, both of a case where generation of speckle data and generation of the control signal for controlling output from the laser light source are executed by a single computer and a case where those processes are executed by different computers. Further, the execution of the respective processes by a predetermined computer includes causing another computer to execute some or all of those processes and acquiring results thereof.

That is, the control method and the program according to the present technology are also applicable to a cloud computing configuration in which one function is shared and cooperatively processed by a plurality of apparatuses via a network.

### Modification

Embodiments of the present technology are not limited to the above-described embodiments and various modifications can be made.

For example, in the above-described embodiments, the image sensor (image capturing section 20) acquires the speckle image from the standard sample irradiated with laser light. Alternatively, it is also possible to use another optical element such as a line sensor or a photodetector (PD) to acquire the speckle image.

In addition, in the above-described embodiments, the control apparatus 30 and the laser driver 14 are configured as different instruments. However, they may be configured as a shared instrument. In addition, the control apparatus 30 and the image capturing section 20 may also be configured as a shared instrument, or the control apparatus 30 and the display section 40 may also be configured as a shared instrument.

In addition, in the above-described embodiments, the brain and the blood vessels of the brain have been described as examples of the observation target M1. However, the present technology is not limited thereto. The present technology is applicable to any living tissue including a part through which scattering material flows such as an organ like a heart, its blood vessels, or its lymph glands in addition to the brain.

In addition, the present technology is applicable to evaluation or the like of an inspection device using a micro flow channel in addition to the living tissue. For example, this makes it possible to detect a flow rate and the like of a solvent flowing through the flow channel, with high accuracy. In addition, fields and the like to which the present technology is applicable are not limited.

Out of the feature parts according to the present technology described above, at least two feature parts can be combined. That is, the various feature parts described in the embodiments may be arbitrarily combined irrespective of the embodiments. Further, various effects described above are merely examples and are not limited, and other effects may be exerted.

Note that, the present technology may also be configured as below.

(1) A control apparatus, including:

a signal generation section that generates speckle data on the basis of an image signal of a subject imaged by using laser light as illumination, and generates a control signal for controlling output from a laser light source that emits the laser light on the basis of the generated speckle data.

(2) The control apparatus according to (1), in which

the subject includes an observation target and a standard sample for calibration, and

the signal generation section generates the speckle data on the basis of an image signal of the standard sample.

(3) The control apparatus according to (1) or (2), further including:

a display control section that causes the display section to display a speckle contrast image of the subject.

(4) The control apparatus according to any one of (1) to (3), in which

in the case where the speckle data is less than a first threshold, the signal generation section generates a control signal for increasing or decreasing the output from the laser light source in a manner that the speckle data becomes the first threshold or more.

(5) The control apparatus according to (4), in which

in the case where the speckle data is less than the first threshold, the signal generation section repeatedly performs control in a manner that the output from the laser light source is increased or decreased by a predetermined amount until the speckle data becomes the first threshold or more, and in the case where an amount of increase or an amount of decrease in the output from the laser light source exceeds a second threshold, the signal generation section generates an error signal.

(6) The control apparatus according to any one of (1) to (5), in which

the speckle data includes speckle contrast, and

the signal generation section generates the control signal on the basis of the speckle contrast.

(7) The control apparatus according to any one of (1) to (5), in which

the image signal includes a plurality of pixel signals, each of which includes luminance information,

the speckle data includes a difference between maximum luminance and minimum luminance, and

the signal generation section generates the control signal on the basis of the difference between the maximum luminance and the minimum luminance.

(8) A control system, including:

an illumination section including a laser light source that emits laser light to an observation target, and a laser driver that adjusts output from the laser light source;

a standard sample for calibration that is capable of being disposed at a position irradiated with the laser light;

an image capturing section that acquires images of the observation target and the standard sample that have been irradiated with the laser light; and

a control apparatus including a signal generation section that generates speckle data from each of pixel signals constituting the image of the standard sample, and generates a control signal for controlling the laser driver on the basis of the generated speckle data.

(9) The control system according to (8), further including:

a display section, in which

the control apparatus further includes a display control section that causes the display section to display a speckle contrast image of the observation target.

(10) The control system according to (8) or (9), in which

the standard sample is a light diffusion optical element.

(11) The control system according to (10), in which

the standard sample is a diffuser plate.

(12) The control system according to (10), in which

the standard sample is a surgical drape.

(13) The control system according to (10) or (11), further including:

a support portion that supports the standard sample, in which

the support portion selectively switches between a first state where the standard sample is disposed in an imaging region of an imaging section and a second state where the standard sample is disposed outside the imaging region of the imaging section.

(14) The control system according to any one of (8) to (13), in which

the imaging section includes a first camera that images the observation target, and a second camera that images the standard sample.

(15) The control system according to any one of (8) to (14), in which

the control system is configured as an endoscope or a microscope.

(16) A control method that is executed by a computer system, the control method including:

generating speckle data on the basis of an image signal of a subject imaged by using laser light as illumination; and

generating a control signal for controlling output from a laser light source that emits the laser light on the basis of the generated speckle data.

(17) A program that causes a computer system to execute:

a step of generating speckle data on the basis of an image signal of a subject imaged by using laser light as illumination; and

a step of generating a control signal for controlling output from a laser light source that emits the laser light on the basis of the generated speckle data.

### REFERENCE SIGNS LIST

10 illumination section
11 laser light source
14 laser driver
20 image capturing section
30 control apparatus
31 signal generation section
32 display control section
40 display section
60 support portion
101, 102, 103, 104, 105 control system
201 first camera
202 second camera
L laser light
M subject
M1 observation target
M2 standard sample

1. A control apparatus, comprising:
a signal generation section that generates speckle data on a basis of an image signal of a subject imaged by using laser light as illumination, and generates a control signal for controlling output from a laser light source that emits the laser light on a basis of the generated speckle data.

2. The control apparatus according to claim 1, wherein
the subject includes an observation target and a standard sample for calibration, and
the signal generation section generates the speckle data on a basis of an image signal of the standard sample.

3. The control apparatus according to claim 1, further comprising:
a display control section that causes the display section to display a speckle contrast image of the subject.

4. The control apparatus according to claim 1, wherein
in a case where the speckle data is less than a first threshold, the signal generation section generates a control signal for increasing or decreasing the output from the laser light source in a manner that the speckle data becomes the first threshold or more.

5. The control apparatus according to claim 4, wherein
in the case where the speckle data is less than the first threshold, the signal generation section repeatedly performs control in a manner that the output from the laser light source is increased or decreased by a predetermined amount until the speckle data becomes the first threshold or more, and in a case where an amount of increase or an amount of decrease in the output from the laser light source exceeds a second threshold, the signal generation section generates an error signal.

6. The control apparatus according to claim 1, wherein
the speckle data includes speckle contrast, and
the signal generation section generates the control signal on a basis of the speckle contrast.

7. The control apparatus according to claim 1, wherein
the image signal includes a plurality of pixel signals, each of which includes luminance information,
the speckle data includes a difference between maximum luminance and minimum luminance, and
the signal generation section generates the control signal on a basis of the difference between the maximum luminance and the minimum luminance.

8. A control system, comprising:
an illumination section including a laser light source that emits laser light to an observation target, and a laser driver that adjusts output from the laser light source;
a standard sample for calibration that is capable of being disposed at a position irradiated with the laser light;
an image capturing section that acquires images of the observation target and the standard sample that have been irradiated with the laser light; and
a control apparatus including a signal generation section that generates speckle data from each of pixel signals constituting the image of the standard sample, and generates a control signal for controlling the laser driver on a basis of the generated speckle data.

9. The control system according to claim 8, further comprising:
a display section, wherein
the control apparatus further includes a display control section that causes the display section to display a speckle contrast image of the observation target.

10. The control system according to claim 8, wherein
the standard sample is a light diffusion optical element.

11. The control system according to claim 10, wherein
the standard sample is a diffuser plate.

12. The control system according to claim 10, wherein
the standard sample is a surgical drape.

13. The control system according to claim 10, further comprising:
a support portion that supports the standard sample, wherein
the support portion selectively switches between a first state where the standard sample is disposed in an imaging region of an imaging section and a second state where the standard sample is disposed outside the imaging region of the imaging section.

14. The control system according to claim 8, wherein
the imaging section includes a first camera that images the observation target, and a second camera that images the standard sample.

15. The control system according to claim 8, wherein
the control system is configured as an endoscope or a microscope.

16. A control method that is executed by a computer system, the control method comprising:
generating speckle data on a basis of an image signal of a subject imaged by using laser light as illumination; and
generating a control signal for controlling output from a laser light source that emits the laser light on a basis of the generated speckle data.

17. A program that causes a computer system to execute:
a step of generating speckle data on a basis of an image signal of a subject imaged by using laser light as illumination; and
a step of generating a control signal for controlling output from a laser light source that emits the laser light on a basis of the generated speckle data.
